(19) United States
(12) Patent Application Publication
(10) Pub. No.: US 2007/0276384 A1
(43) Pub. Date: Nov. 29, 2007

(54) Hook for Fixing a Spinal Support Rod to a Vertebra

(76) Inventor: Frank Spratt, Blyth (GB)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/579,704

(22) PCT Filed: Nov. 17, 2004
(86) PCT No.: PCT/GB04/04845

§ 371 Date: May 11, 2007

(30) Foreign Application Priority Data
Nov. 19, 2003 (GB) 0326888.5

(51) Int. Cl. A61B 17/58

(52) U.S. Cl. 606/72; 606/60; 606/73

(57) ABSTRACT

A hook for joining a vertebra and a rod, comprising a main body (4) with a channel portion (10) and a hook portion (8). The channel portion (10) is for receiving a spinal support rod, and the hook portion (8) is for receiving vertebral tissue. The hook incorporates a clamp (6) which rotates about an axis so that a sharp tip of the clamp (6) can move towards and away from the free end of the hook portion, allowing a bone to be gripped within the hook portion (8) by the clamp (6).

The present invention relates to hooks for fixing a spinal support rod to a vertebra.

It is known to attach rods to the bones of the spine for use in correcting spinal defects. Typically the rod is connected to the spine using a hook which engages a portion of the vertebra, for example the pedicle or the transverse process. There exists a need to secure vertebral tissue within the hook in order to ensure that the rod remains correctly in place both during and after surgery.

One way to secure a hook is to use a screw which penetrates into the bone, holding the vertebra securely within the hook. For example, FR-A-2642642 (Cotrel) shows a hook in which a screw passes through a hole drilled in the bone substantially perpendicular to the tip of the hook. U.S. Pat. No. 5,584,832 (Schläpfer) also shows a hook secured using a screw. However, unlike Cotrel, Schläpfer uses a screw inclined at an angle to the tip of the hook. During surgery using the hooks shown in both Schläpfer and Cotrel, it is necessary to drill a hole in the bone to receive the screw. This ensures that the screw is correctly positioned and also limits any splintering of the bone which could occur if the screw was used without pre-drilling. This adds an additional step into surgery and also introduces a further point at which a mistake could be made. In both of the systems, because a hole must be drilled in the bone, it is difficult to make fine adjustments to the position of a hook once a hole has been drilled. Coarse adjustments are possible, but only when the next hole is located a sufficient distance from the previous hole to ensure there is no risk of the drilled hole causing the bone tissue to be weakened unacceptably.

The present invention provides a hook with a channel for receiving a spinal support rod, a hook for receiving a vertebral tissue and a clamp with a sharp tip for clamping the vertebral tissue in place within the hook.

Accordingly, in one aspect, the present invention provides a hook for fixing a spinal support rod to a vertebra by engaging the vertebra, which has a hook portion with a recess in which vertebral tissue can be received, and a fixation portion which includes a channel in which a spinal support rod can be received, the hook including a clamp having a sharp tip which can penetrate the surface of the vertebra when it is located in the hook portion recess, in which the clamp is pivotally fixed to the hook and can be displaced between a clamping position in which it engages the surface of the vertebra to inhibit removal of the vertebra from within the recess, and a release position in which vertebral tissue can be inserted into and removed from the recess.

The tip should be capable of being driven into a surface purely by applying a force directed through the sharp tip into the surface. The tip should be capable of being driven into a bone surface without any twisting, such as is necessary when a screw is used to fix a hook to the bone. For example, the tip can be conical. The tip can be provided by a plurality of conical points. The tip can have the form of a cutting edge, for example by being provided by a block with a constant cross-section (“prismatic”). The tip can be provided by a plurality of cutting edges, for example arranged in parallel. The tip can be described as “wedge-shaped”.

Use of a sharp tip, especially if it provides an elongate cutting edge (straight or non-straight), has the advantage that it can allow the clamp to be repositioned with less tendency for the tip to “slip” back into a previous indentation compared with a hook which relies on a screw for fixation. Particularly fine adjustment is available in systems using a cutting edge. In such a system very fine adjustments can be made in the direction of the edge, by extending the existing indentation slightly at one end.

With the clamp in the release position vertebral tissue can be inserted into the recess. Once the vertebra is in position within the recess the clamp can be moved to the clamping position in which the sharp tip penetrates the surface of the bone, to retain the bone within the hook. If repositioning is required the clamp can be moved back towards the release position allowing the bone to be moved freely relative to the hook. The clamping member has a sharp end and therefore can secure the bone within the hook.

Preferably, the displacement of the tip between the clamping position and the release position is less than 5 mm, more preferably less than 3 mm. This range of displacement allows the tip to bite into the vertebral tissue sufficiently to hold the tissue securely within the recess.

Preferably, the tip can move in a clamping direction towards the clamping position, and can penetrate the surface of the vertebral tissue solely by the application of a force to the clamp in the clamping direction. The hook can therefore be installed simply by pressing the clamp into the clamping position. There is also no need to apply a rotational torque or twisting to the clamp about the clamping direction, as there is in existing systems which use a screw to secure bone tissue in a hook. This gives simpler installation than screw systems, where it can be difficult to access and turn the screw.

Preferably, the width of the recess in which the bone is received is smaller when the clamp is in the clamping position than when the clamp is in the release position. Bone which is received in the recess is therefore clamped between the clamp and the opposing surface of the recess, and held in place as a result of penetration of the surface of the bone by the tip.

Preferably, the hook further comprises an actuator for displacing the sharp tip of the clamp towards the clamping position. This allows the movement of the tip towards the clamping position to be finely controlled. A surgeon can control the extent of the penetration of the tip into the bone tissue through use of the actuator. Further, once the hook has been secured in the correct position, the presence of the actuation element ensures that the hook cannot accidentally move back towards the first position, to provide a secure grip of the bone within the hook. Another advantage is that the actuator can be arranged to be in a position which can be accessed easily by the surgeon.

Preferably, the actuator is a grub screw. This allows the clamp to be moved towards the second position with fine control over its position, depending on the pitch of the grub screw. The length of-the grub screw can be chosen so that when the clamp is at the clamping position, the grub screw is entirely enclosed within the body of the hook, ensuring that there is not an unnecessary projection or sharp edge.

Preferably, the actuator is operated from within the channel. Preferably, location of a spinal support rod within the channel prevents access to the actuator.

Preferably, the actuator is provided by the spinal support rod which can be received in the channel. The spinal support rod can engage the clamp (directly or indirectly) when it is located in the channel to cause the tip to penetrate the surface of the bone as a result of the application of force to the rod.

Preferably, the hook further comprises a stop portion which abuts the clamp when it is in the clamping position. The stop portion ensures that the sharp tip cannot be driven too far into a bone by mistake. This allows a surgeon to know that the tip has penetrated the bone sufficiently to hold the hook securely, and at the same time ensure that the sharp tip is not driven too far into the bone to cause adverse side effects.

Preferably, the tip of the clamp is a single point or a single sharp edge. This allows the clamping member to grip the bone securely.

In one embodiment of the invention, the spinal support rod has a longitudinal axis, and the clamp is pivotally fixed to the hook such that the clamp can pivot about an axis between the channel and the hook portion, transverse to a plane intersecting the longitudinal axis and the hook portion. This construction allows the clamp to be incorporated within the body of the hook. It also ensures that in use the clamping member does not interfere with the rod installed in the channel. A further advantage is that the pivotal connection allows a lever action to adjust the force required to displace the tip, by altering the distance from the pivot at which the clamping force acts.

In another embodiment, a side of the channel adjacent to the recess is open, such that when a rod is installed in the channel the rod abuts, and exerts a force on, the clamp, through direct or indirect contact with the clamp. This means that during assembly of the hook together with a rod, the rod itself acts on the clamp (directly or indirectly) to press it into position. It is therefore easy to adjust both the position of the rod in the channel and vertebral tissue in the recess simultaneously. Optionally, the surface of the clamp adjacent to the channel is inclined. This allows the position at which the force is applied to the clamp to be varied as the rod is tightened into position, giving fine control over the force profile require to displace the tip from the release to the clamping position.

In a further aspect, the invention provides a system for joining a rod to a bone comprising a spinal support rod and a hook as discussed above.

Embodiments of the invention will now be described, by way of example only, with reference to the following drawings in which:

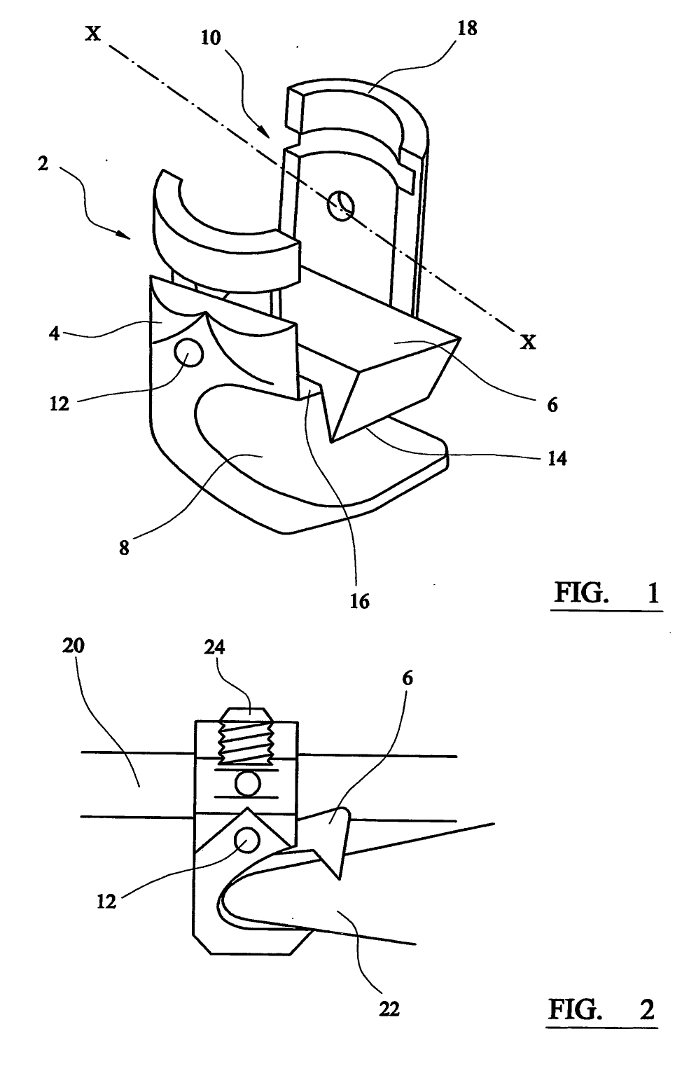

FIG. 1 is a perspective view of a first embodiment of the present invention;

FIG. 2 is a side view of the hook of the first embodiment;

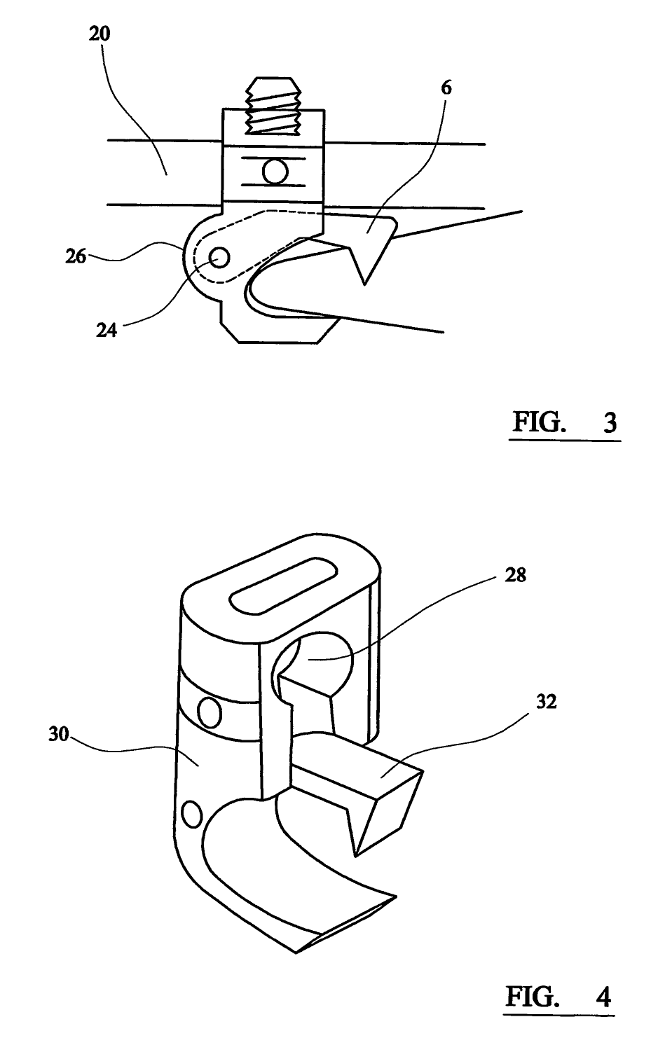

FIG. 3 is a side view of a second embodiment of the hook of the present invention; and

FIG. 4 is a perspective view of a fourth embodiment of the hook of the present invention.

FIG. 1 depicts a perspective view of a first embodiment of the present invention. The hook 2 comprises a main body 4 and a clamping member or clamp 6 pivotally attached to the main body 4. The main body 4 is formed as a single piece from, for example titanium, and incorporates a lower hook portion with a recess 8 and an upper fixing portion with a channel 10. The dimensions of the channel 10 are chosen such that a spinal support rod (not illustrated) for use in spinal surgery can be accommodated within the channel 10. This rod has a longitudinal axis XX.

The clamp 6 is located below the channel 10 and is pivotally connected to the main body 4 by pins 12. Two holes are formed in the main body 4, between the recess 8 and the channel 10, to receive the pins 12. This defines an axis transverse to the longitudinal axis XX of the rod, about which the clamp 6 can pivot. The clamp 6 has a sharp tip 14 which faces the free end of the hook portion. Thus, the sharp end 14 of the clamp 6 can move towards and away from the free end of the hook portion, between a clamping position and a release position to allow insertion of vertebral tissue, for example the pedicle or the transverse process, into the recess.

The recess 8 is shaped such that it can receive vertebral tissue, the free end of the hook portion extending in a direction which is approximately parallel to the longitudinal axis XX of the rod.

The main body 4 incorporates a shelf 16 which acts as a stop for the clamp 6, preventing movement of the sharp end 14 too close to the free end of the hook portion.

The channel 10 has a lower surface defined by :the upper surface of the clamp 6. When a rod is placed in the channel 10 moving the rod in a direction towards the recess hook portion causes the clamp 6 to move its sharp end 14 towards the clamping position. The surface of the clamp 6 adjacent to the channel is inclined, so that the point at which the rod exerts a force on the clamp alters as the rod is moved towards the hook portion. A screw thread 18 is incorporated in the upper end of the channel for receiving a screw to hold the rod in place within the channel 10.

FIG. 2 shows a side view of the hook 2 when installed with a rod 20 and a bone 22. Installation of the rod 20 in the channel 10 exerts a force on the inclined face of the clamp 6. This causes the sharp end 14 of the clamp 6 to move towards the clamping position and bite into the bone. The rod 20 is tightened into position by a grub screw 24.

During surgery, the surgeon initially positions the bone 22 and rod 20 together with the hook 2 by loosely tightening the grub screw 24. This causes the sharp end 14 of the clamp 6 to bite into the bone tissue whilst at the same time securing the rod 20 into place in the channel. If desired, the surgeon can loosen the grub screw 24 to allow repositioning of the rod 20 and the bone 22 as required. When the correct position is reached, the surgeon tightens the grub screw 24 until the clamp 6 abuts the stop element 16. This ensures that the clamp 6 is sufficiently engaged with the bone 22 to hold it securely in place. It can therefore be straightforward for the surgeon to adjust the position of both the hook and the bone using only the grub screw 24.

The sharp end 14 of the clamp 6 comprises a single sharp edge. This allows the surgeon to carry out initial positioning of the bone within the recess 8. The clamp 6 bites sufficiently into the tissue to enable the surgeon to determine whether the correct positioning has been achieved. The use of this sharp edge also ensures that, even when the clamping member has been screwed into the bone to a distance at which it abuts the stop portion 16, the grub screw 24 can be loosened and repositioning of the rod 20 and bone 22 achieved easily. There is no need to drill a hole prior to securing the bone in the hook. Should subsequent repositioning be required this can be carried out easily. Further, the use of a sharp edge reduces splintering of the bone, and allows fine repositioning to be carried out with no difficulty.

Whilst the above embodiment has described the use of a grub screw, other types of screw could also be used. For example, in an alternative construction, a screw with a head could be used in which case the position in which the head of the screw abuts the top of the body can define the stop position. In such an alternative construction, there is no need to provide a stop element 16 under the clamp 6.

Although the present embodiment is manufactured from titanium, any other bio-compatible metal or other material, such as a thermoplastic or other polymer, could also be used.

FIG. 3 depicts a side view of a second embodiment of the present invention. The construction of this embodiment is the same as the first, save as described below.

In this embodiment, the pins 24 connecting the clamp to the body of the hook are moved towards the rear of the hook, roughly opposite the position of the tip of the bone when it is held within the recess. This gives a lever action, such that the tip of the clamp 6 moves a greater distance than the actuation element and allows the use of the smaller actuation element.

The movement of the pins 24 to this new position requires additional reinforcement 26 to be provided at the rear of the hook. This stops the pins 24 tearing through the rear of the hook body when the apparatus is tightened.

FIG. 4 illustrates a perspective view of a third embodiment of the present invention. In this embodiment the channel 28 for receiving the rod is formed entirely from the main body of the hook. When a rod is installed in the channel 28, it does not touch the clamp 32. An actuation element, such as a grub screw (not illustrated) acts directly on the clamp 32 to allow tightening of the clamping member onto a bone positioned in the recess.

This embodiment allows the tightening of the clamp 32 to be adjusted independently of the installation of a rod in the channel 28.

Although all of the above embodiments have used a clamp which is pivotally connected to the body of the hook, the clamp could also be directly displaced into the bone by the action of a force. For example, the clamp could be a rod with a sharp tip at one end which is secured into the bone by pressing on the other end.

1. A hook for fixing a spinal support rod to a vertebra by engaging the vertebra, which has a hook portion with a recess in which vertebral tissue can be received, and a fixation portion which includes a channel in which a spinal support rod can be received, the hook including a clamp having a sharp tip which can penetrate the surface of the vertebra when it is located in the hook portion recess, in which the clamp is pivotally fixed to the hook and can be displaced between a clamping position in which it engages the surface of the vertebra to inhibit removal of the vertebra from within the recess, and a release position in which vertebral tissue can be inserted into and removed from the recess.

2. A hook as claimed in claim 1, in which the displacement of the tip between the clamping position and the release position is less than 5 mm, preferably less than 3 mm.

3. A hook as claimed in claim 1, wherein the width of the recess in which the bone is received is smaller when the clamp is in the clamping position than when the clamp is in the release position.

4. A hook as claimed in claim 1, further comprising an actuator for displacing the clamp towards the clamping position.

5. A hook as claimed in claim 4, wherein the actuator comprises a grub screw.

6. A hook as claimed in claim 1, further comprising a stop portion which abuts the clamp when it is in the clamping position.

7. A hook as claimed in claim 1, wherein the sharp end of the clamping member is a single point or a single sharp edge.

8. A hook as claimed in claim 1, in which the clamp can pivot about an axis between the channel and the hook portion, transverse to a plane intersecting the axis of the support rod when located in the channel and the hook portion.

9. A hook as claimed in claim 1, wherein a side of the channel adjacent to the recess is open, such that when a rod is installed in the channel the rod acts on the clamp directly or indirectly to cause it to penetrate the bone.

10. A hook as claimed in claim 9, wherein the surface of the clamp which is adjacent to the channel is inclined.

11. A system for joining a rod to a bone comprising:
a spinal support rod; and
a hook for fixing a spinal support rod to a vertebra by engaging the vertebra, which has a hook portion with a recess in which vertebral tissue can be received, and a fixation portion which includes a channel in which the spinal support rod can be received, the hook including a clamp having a sharp tip which can penetrate the surface of the vertebra when it is located in the hook portion recess, in which the clamp is pivotally fixed to the hook and can be displaced between a clamping position in which it engages the surface of the vertebra to inhibit removal of the vertebra from within the recess, and a release position in which vertebral tissue can be inserted into and removed from the recess.
